(19) United States
(12) Patent Application Publication
(10) Pub. No.: US 2011/0118783 A1
(43) Pub. Date: May 19, 2011

(54) LOAD-SHARING BONE ANCHOR HAVING A FLEXIBLE POST AND METHOD FOR DYNAMIC STABILIZATION OF THE SPINE

(75) Inventors: Charles J. Winslow, Walnut Creek, CA (US); Steven T. Mitchell, Pleasant Hill, CA (US); John J. Flynn, Walnut Creek, CA (US); James F. Zucherman, San Francisco, CA (US); Ken Y. Hsu, San Francisco, CA (US); Henry A. Klyce, Piedmont, CA (US); H. Adam R. Klyce, Berkeley, CA (US)

(73) Assignee: Spartek Medical, Inc., Alameda, CA (US)

(21) Appl. No.: 12/898,133

(22) Filed: Oct. 5, 2010

Related U.S. Application Data
(60) Provisional application No. 61/261,545, filed on Nov. 16, 2009.

(51) Int. Cl. A61B 17/70

(52) U.S. Cl. 606/257; 606/264

(57) ABSTRACT

A dynamic stabilization system including a flexible bone anchor and methods for assembling a dynamic stabilization assembly which supports the spine while providing for the preservation of spinal motion. The flexible bone anchor includes a flexible post mounted within a bone anchor. Deflection of the flexible post is controlled by a flexible section integrated into the flexible post. A housing encloses the flexible post isolating it from the bone and providing a stable connection point for other elements of the implant. An internal surface within the housing is positioned to limit deflection of the flexible post. The force/deflection properties of the flexible bone anchor are adapted to be configured and/or customized to the anatomy and functional requirements of the patient by changing the properties of the flexible section and housing.

### CLAIM TO PRIORITY

This application claims priority to the following patents and patent applications:

U.S. Provisional Application No. 61/261,545, filed Nov. 16, 2009, entitled “LOAD-SHARING BONE ANCHOR HAVING A FLEXIBLE POST AND METHOD FOR DYNAMIC STABILIZATION OF THE SPINE” (Attorney Docket No. SPART-01050US0).

All of the afore-mentioned patent applications are incorporated herein by reference in their entireties.

### CROSS-REFERENCES TO RELATED APPLICATIONS

This application is related to all of the afore-mentioned patent applications. This application is also related to all of the following applications including:

U.S. patent application Ser. No. 12/566,487, filed Sep. 24, 2009, entitled “Versatile Offset Polyaxial Connector And Method For Dynamic Stabilization Of The Spine” (Attorney Docket No. SPART-01043US2); and

U.S. patent application Ser. No. 12/566,491, filed Sep. 24, 2009, entitled “Load-Sharing Bone Anchor Having A Deflectable Post And Method For Dynamic Stabilization Of The Spine” (Attorney Docket No. SPART-01044US1); and

U.S. patent application Ser. No. 12/566,494, filed Sep. 24, 2009, entitled “Load-Sharing Component Having A Deflectable Post And Method For Dynamic Stabilization Of The Spine” (Attorney Docket No. SPART-01044US5); and

U.S. patent application Ser. No. 12/566,498, filed Sep. 24, 2009, entitled “Load-Sharing Bone Anchor Having A Durable Compliant Member And Method For Dynamic Stabilization Of The Spine” (Attorney Docket No. SPART-01044US6); and

U.S. patent application Ser. No. 12/566,504, filed Sep. 24, 2009, entitled “Load-Sharing Bone Anchor Having A Deflectable Post With A Compliant Ring And Method For Stabilization Of The Spine” (Attorney Docket No. SPART-01044US7); and

U.S. patent application Ser. No. 12/566,507, filed Sep. 24, 2009, entitled “Load-Sharing Bone Anchor Having A Deflectable Post With A Compliant Ring And Method For Stabilization Of The Spine” (Attorney Docket No. SPART-01044US8); and

U.S. patent application Ser. No. 12/566,511, filed Sep. 24, 2009, entitled “Load-Sharing Bone Anchor Having A Deflectable Post And Method For Stabilization Of The Spine” (Attorney Docket No. SPART-01044US9); and

U.S. patent application Ser. No. 12/566,516, filed Sep. 24, 2009, entitled “Load-Sharing Bone Anchor Having A Natural Center Of Rotation And Method For Dynamic Stabilization Of The Spine” (Attorney Docket No. SPART-01044USA); and

U.S. patent application Ser. No. 12/566,519, filed Sep. 24, 2009, entitled “Dynamic Spinal Rod And Method For Dynamic Stabilization Of The Spine” (Attorney Docket No. SPART-01044USC); and

U.S. patent application Ser. No. 12/566,522, filed Sep. 24, 2009, entitled “Dynamic Spinal Rod Assembly And Method For Dynamic Stabilization Of The Spine” (Attorney Docket No. SPART-01044USD); and

U.S. patent application Ser. No. 12/566,529, filed Sep. 24, 2009, entitled “Configurable Dynamic Spinal Rod And Method For Dynamic Stabilization Of The Spine” (Attorney Docket No. SPART-01044USE); and

U.S. patent application Ser. No. 12/566,531, filed Sep. 24, 2009, entitled “A Spinal Prosthesis Having A Three Bar Linkage For Motion Preservation And Dynamic Stabilization Of The Spine” (Attorney Docket No. SPART-01044USF); and

U.S. patent application Ser. No. 12/566,534, filed Sep. 24, 2009, entitled “Surgical Tool And Method For Implantation of A Dynamic Bone Anchor” (Attorney Docket No. SPART-01045US1); and

U.S. patent application Ser. No. 12/566,547, filed Sep. 24, 2009, entitled “Surgical Tool And Method For Connecting A Dynamic Bone Anchor and Dynamic Vertical Rod” (Attorney Docket No. SPART-01045US2); and

U.S. patent application Ser. No. 12/566,551, filed Sep. 24, 2009, entitled “Load-Sharing Bone Anchor Having A Deflectable Post And Centering Spring And Method For Dynamic Stabilization Of The Spine” (Attorney Docket No. SPART-01049US1); and

U.S. patent application Ser. No. 12/566,553, filed Sep. 24, 2009, entitled “Load-Sharing Component Having A Deflectable Post And Centering Spring And Method For Dynamic Stabilization Of The Spine” (Attorney Docket No. SPART-01049US2); and

U.S. patent application Ser. No. 12/566,559, filed Sep. 24, 2009, entitled “Load-Sharing Bone Anchor Having A Deflectable Post And Axial Spring And Method For Dynamic Stabilization Of The Spine” (Attorney Docket No. SPART-01053US1); and

U.S. patent application Ser. No. 12/629,811, filed Dec. 2, 2009, entitled “Low Profile Spinal Prosthesis Incorporating a Bone Anchor Having a Deflectable Post and a Compound Spinal Rod” (Attorney Docket No. SPART-01057US1).

All of the afore-mentioned patent applications are incorporated herein by reference in their entireties.

### BACKGROUND OF INVENTION

Back pain is a significant clinical problem and the costs to treat it, both surgical and medical, are estimated to be over $2 billion per year. One method for treating a broad range of degenerative spinal disorders is spinal fusion. Implantable medical devices designed to fuse vertebrae of the spine to treat have developed rapidly over the last decade. However, spinal fusion has several disadvantages including reduced range of motion and accelerated degenerative changes adjacent the fused vertebrae.

Alternative devices and treatments have been developed for treating degenerative spinal disorders while preserving motion. These devices and treatments offer the possibility of treating degenerative spinal disorders without the disadvantages of spinal fusion. However, current devices and treatments suffer from disadvantages e.g., complicated implantation procedures; lack of flexibility to conform to diverse patient anatomy; the need to remove tissue and bone for implantation; increased stress on spinal anatomy; insecure anchor systems; poor durability, and poor revision options. Consequently, there is a need for new and improved devices and methods for treating degenerative spinal disorders while preserving motion.

### SUMMARY OF INVENTION

The present invention includes a spinal implant system and methods that can dynamically stabilize the spine while providing for the preservation of spinal motion. Embodiments of the invention provide a dynamic stabilization system which includes: versatile components, adaptable stabilization assemblies, and methods of implantation. An aspect of embodiments of the invention is the ability to stabilize two, three and/or more levels of the spine. Another aspect of embodiments of the invention is the ability to select components of embodiments of the invention which are appropriate to the anatomy and functional requirements of a patient. Another aspect of embodiments of the invention is the ability to accommodate particular anatomy of the patient by providing a system of versatile components which is adaptable to the anatomy and needs of a particular patient and procedure. Another aspect of the invention is to facilitate the process of implantation and minimize disruption of tissues during implantation.

Thus, the present invention provides new and improved systems, devices and methods for treating degenerative spinal disorders by providing and implanting a dynamic spinal stabilization assembly which supports the spine while preserving motion. These and other objects, features and advantages of the invention will be apparent from the drawings and detailed description which follow.

### BRIEF DESCRIPTION OF THE DRAWINGS

FIG. 1A is a perspective view of a bone anchor having a flexible post according to an embodiment of the present invention.

FIG. 1B is a perspective view of a connector mounted to the bone anchor of FIG. 1A according to an embodiment of the present invention.

FIG. 1C is an exploded view of a dynamic vertical rod.

FIG. 1D is a perspective view of the dynamic vertical rod of FIG. 1C connector mounted to the bone anchor of FIG. 1A according to an embodiment of the present invention.

FIG. 1E is a posterior view of a multi-level dynamic stabilization implant utilizing the components of FIGS. 1A to 1D according to an embodiment of the present invention.

FIG. 1F is a lateral view of a multi-level dynamic stabilization assembly utilizing the components of FIGS. 1A to 1D according to an embodiment of the present invention.

FIG. 2A is an exploded view of a flexible bone anchor according to an embodiment of the present invention.

FIG. 2B is an enlarged view of the flexible post of the flexible bone anchor of FIG. 2A according to an embodiment of the present invention.

FIG. 2C is a sectional view of the flexible bone anchor of FIG. 2A as assembled.

FIG. 2D is a sectional view of the flexible bone anchor of FIG. 2A as assembled and illustrating deflection of the flexible post under load.

FIGS. 3A-3D show alternative flexible posts for flexible bone anchors according to embodiments of the present invention.

FIG. 4A is an exploded view of an alternative flexible bone anchor according to an embodiment of the present invention.

FIG. 4B is a perspective view of the alternative flexible bone anchor of FIG. 4A.

FIG. 4C is a sectional view of the alternative flexible bone anchor of FIG. 4A as assembled.

FIG. 4D is a sectional view of the alternative flexible bone anchor of FIG. 4A as assembled and illustrating deflection of the flexible post under load.

FIG. 5A is an exploded view of an alternative flexible bone anchor according to an embodiment of the present invention.

FIG. 5B is a perspective view of the flexible post of the alternative flexible bone anchor of FIG. 5A according to an embodiment of the present invention.

FIG. 5C is a sectional view of the alternative flexible bone anchor of FIG. 5A as assembled.

FIG. 5D is a sectional view of the alternative flexible bone anchor of FIG. 5A as assembled and illustrating deflection of the flexible post under load.

FIG. 5E is a sectional view of another alternative flexible bone anchor as assembled.

FIG. 5F is a sectional view of another alternative flexible bone anchor as assembled.

FIGS. 6A-6F show alternative flexible posts for flexible bone anchors according to embodiments of the present invention.

FIGS. 7A-7E are perspective views of alternative combinations of flexible bone anchors and bone anchors according to embodiments of the present invention.

### DETAILED DESCRIPTION OF THE PREFERRED EMBODIMENTS

The present invention includes a versatile spinal implant system and methods which can dynamically stabilize the spine while providing for the preservation of spinal motion. Alternative embodiments can be used for spinal fusion. An aspect of the invention is restoring and/or preserving the natural motion of the spine including the quality of motion as well as the range of motion. Still, another aspect of the invention is providing for load sharing and stabilization of the spine while preserving motion.

Another aspect of the invention is to provide a modular system which can be customized to the needs of the patient. Another aspect of embodiments of the invention is the ability to stabilize two, three and/or more levels of the spine by the selection of appropriate components for implantation in a patient. Another aspect of the invention is the ability to provide for higher stiffness and fusion at one level or to one portion of the spine while allowing for lower stiffness and dynamic stabilization at another adjacent level or to another portion of the spine. Embodiments of the invention allow for fused levels to be placed next to dynamically-stabilized levels. Such embodiments of the invention enable vertebral levels adjacent to fusion levels to be shielded by providing a transition from a rigid fusion level to a dynamically stable, motion preserved, and more mobile level.

Embodiments of the present invention provide for assembly of a dynamic stabilization system which supports the spine while providing for the preservation of spinal motion. The dynamic stabilization system includes an anchor system, a vertical rod system and a connection system. The anchor system anchors the construct to the spinal anatomy and includes flexible bone anchors and conventional bone anchors. The deflection system provides dynamic stabilization while reducing the stress exerted upon the bone anchors and spinal anatomy. The vertical rod system connects different levels of the construct in a multilevel assembly and may in some embodiments include compound flexible bone anchors. The connection system includes coaxial connectors and offset connectors which adjustably connect the deflection system, vertical rod system and anchor system allowing for appropriate, efficient and convenient placement of the anchor system relative to the spine. Alternative embodiments can be used for spinal fusion.

Embodiments of the invention include a construct with an anchor system, a vertical rod system and a connection system. The anchor system includes flexible bone anchors which provide dynamic stabilization while reducing the stress exerted upon the bone anchors and spinal anatomy. The connection system connects the anchor system to the vertical rod system. The vertical rod system connects dynamic stabilization system components on different vertebra to provide load sharing and dynamic stabilization.

Embodiments of the present invention include a flexible bone anchor which provides load sharing while preserving range of motion and reducing stress exerted upon the bone anchors and spinal anatomy. The flexible bone anchor includes a flexible post mounted within a bone anchor. Deflection of the flexible post is controlled by a flexible section integrated into the flexible post. A contact surface of the bone anchor is positioned to limit deflection of the flexible post. In some embodiments of the present invention the force/deflection properties of the flexible bone anchor are adapted and/or customized to the anatomy and functional requirements of the patient by changing the properties of the flexible post and/or flexible section. Different flexible bone anchors having different force/deflection properties are adapted to be utilized in different patients or at different spinal levels within the same patient depending upon the anatomy and functional requirements.

Common reference numerals are used to indicate like elements throughout the drawings and detailed description; therefore, reference numerals used in a drawing may or may not be referenced in the detailed description specific to such drawing if the associated element is described elsewhere. The first digit in a reference numeral indicates the series of figures in which the referenced item first appears.

The terms “vertical” and “horizontal” are used throughout the detailed description to describe general orientation of structures relative to the spine of a human patient that is standing. This application also uses the terms proximal and distal in the conventional manner when describing the components of the spinal implant system. Thus, proximal refers to the end or side of a device or component closest to the hand operating the device, whereas distal refers to the end or side of a device furthest from the hand operating the device. For example, the tip of a bone screw that enters a bone would conventionally be called the distal end (it is furthest from the surgeon) while the head of the screw would be termed the proximal end (it is closest to the surgeon).

### Dynamic Stabilization System

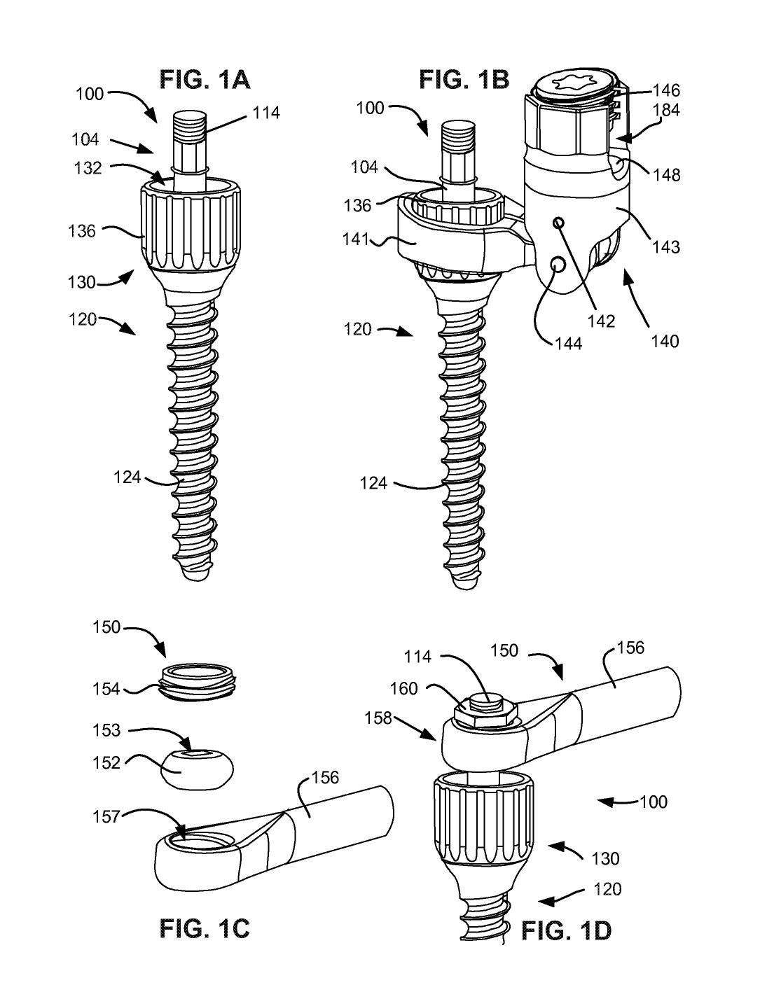

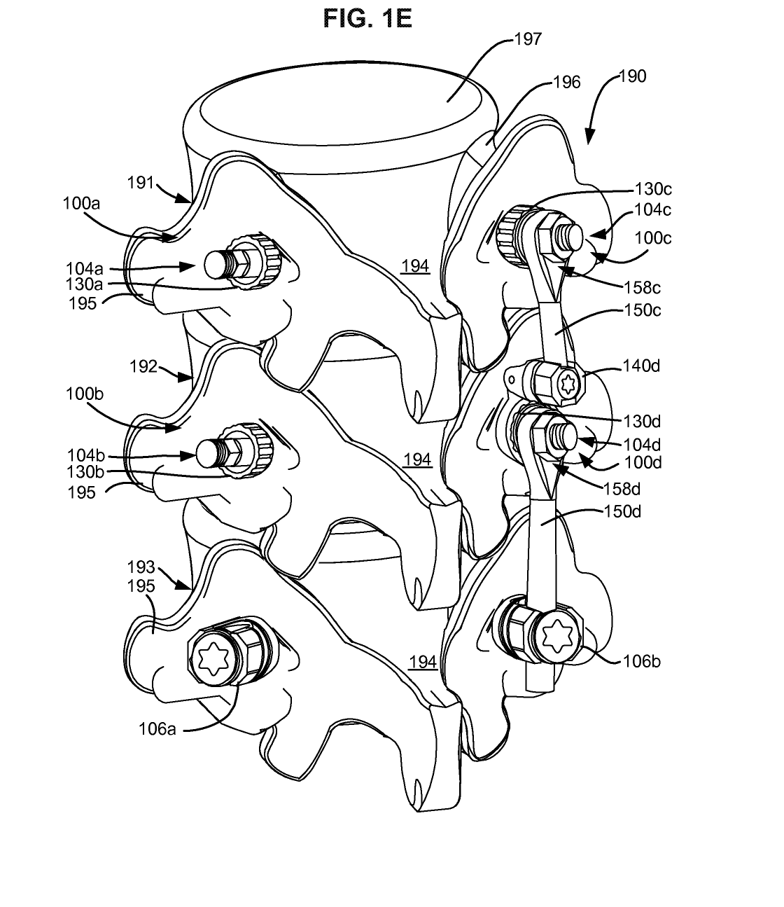

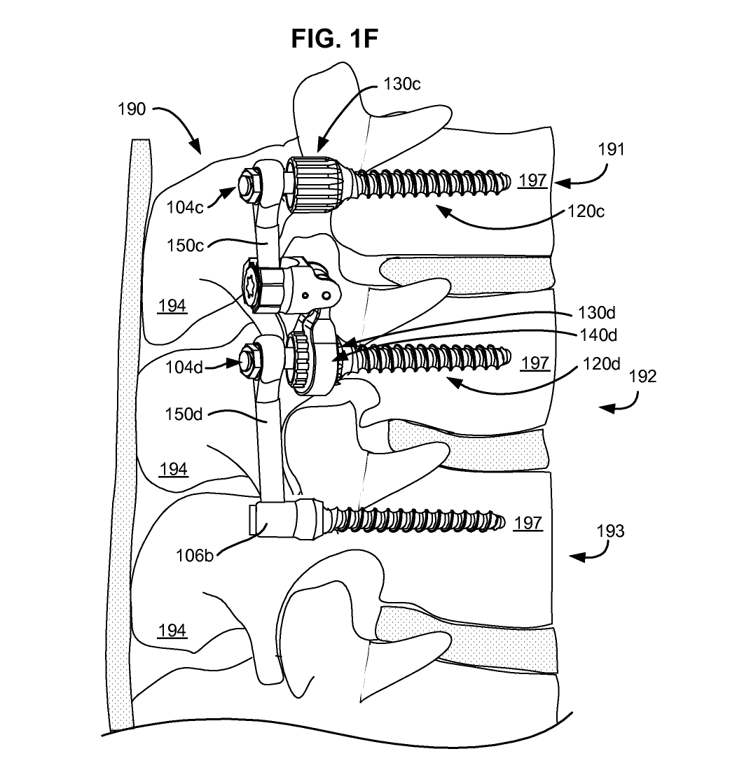

FIGS. 1A-1F introduce components of a dynamic stabilization system according to an embodiment of the present invention. The components include anchor system components, vertical rods and connection system components, including for example coaxial and offset connectors. The components are implanted and assembled to form a dynamic stabilization system appropriate for the anatomical and functional needs of a patient.

FIG. 1A shows a flexible bone anchor 100. Flexible bone anchor 100 is a bone anchor having controlled flexibility which allows for load sharing. The flexible bone anchor 100 provides stiffness and support where needed to support the loads exerted on the spine during normal spine motion, which loads, the soft tissues of the spine are no longer able to accommodate since these spine tissues are either degenerated or damaged. Load sharing is enhanced by the ability to select the appropriate stiffness of the flexible bone anchor 100 in order to match the load sharing characteristics desired.

Flexible bone anchor 100 includes a bone screw 120. Bone screw 120 has a threaded shaft 124 and a housing 130. Housing 130 has a bore 132 coaxial with the longitudinal axis of bone screw 120. Bore 132 is adapted to receive a flexible post 104. Threaded shaft 124 is adapted to engage a bone to secure the flexible bone anchor 100 onto a bone. The flexible bone anchor 100 may alternatively include one or more alternative bone anchors known in the art e.g. bone hooks, expanding devices, barbed devices, threaded devices, adhesive and other devices capable of securing a component to bone instead of or in addition to threaded shaft 124.

A flexible post 104 extends from the proximal end of cavity 132. The proximal end of flexible post 104 includes a mount 114 for connecting a vertical rod. Mount 114 may deflect in a controlled manner relative to bone anchor 120 by bending of flexible post 104. The bending of flexible post 104 and deflection of mount 114 relative to bone anchor 120 provides for load sharing and motion preservation. The stiffness/flexibility of deflection of the flexible post 104 may be controlled and/or customized as will be described below. Flexible post 104 is attached at its distal end to the bone anchor 120 in the bottom of bore 132. The distal end of flexible post 104 is configured to be attached to bone anchor 120 by threads and/or alternative mechanisms and techniques, including, for example, welding, soldering, bonding, and/or mechanical fittings including threads, snap-rings, locking washers, cotter pins, bayonet fittings or other mechanical joints.

As shown in FIG. 1A, flexible post 104 is oriented in a substantially co-axial, collinear or parallel orientation to bone anchor 120. This arrangement simplifies implantation, reduces trauma to structures surrounding an implantation site, and reduces system complexity. Arranging the flexible post 104 co-axial with the bone anchor 120 can substantially transfer a moment (of) force applied by the flexible post 104 from a moment force tending to pivot or rotate the bone anchor 120 about the axis of the shaft, to a moment force tending to act perpendicular to the axis of the shaft. The flexible bone anchor 100 can thereby effectively resist repositioning of the bone anchor 120 without the use of locking screws or horizontal bars to resist rotation. Further examples of flexible bone anchors are provided below. Each of the flexible bone anchors described herein is adapted to be used as a component of a dynamic stabilization system.

Flexible bone anchor 100 also preferably includes a coupling surface 136 to which other components are adapted to be mounted. As shown in FIG. 1A, coupling surface 136 is the external cylindrical surface of housing 130. Flexible bone anchor 100 thus provides two mounting positions, one being the mount 114 of flexible post 104 (a coaxial mounting position) and one being the coupling surface 136 (an external or offset mounting position). Thus a single flexible bone anchor 100 can serve as the mounting point for one, two or more components. For example, a vertical rod is adapted to be mounted to mount 114 and a component of the connection system is adapted to be mounted to the coupling surface 136 of the housing 130 (See, e.g. FIG. 1B). As shown in FIG. 1B, mount 114 can deflect relative to bone anchor 120 whereas coupling surface 136 is fixed relative to bone anchor 120. Moreover, housing 130 extends over flexible post 104 to isolate moving parts of flexible bone anchor 100 from the bone. In some embodiments, the flexible bone anchor is adapted to be implanted such that a deflectable portion of flexible post 104 is at or below the surface of the bone.

FIG. 1B shows a component of the connection system which is adapted to be mounted to the coupling surface 136 of the housing 130 of flexible bone anchor 100. FIG. 1B shows a perspective view of offset connector 140 mounted externally to housing 130 of flexible bone anchor 100. Connector 140 may be termed an offset head or offset connector. Offset connector 140 comprises six components and allows for two degrees of freedom of orientation and two degrees of freedom of position in connecting a vertical rod to a bone anchor. The six components of offset connector 140 are dowel pin 142, pivot pin 144, locking set screw 146, plunger 148, clamp ring 141 and saddle 143. Saddle 143 has a slot 184 sized to receive a rod, for example a vertical rod e.g. vertical rod 106 of FIG. 1A. Locking set screw 146 is mounted at one end of slot 184 such that it is adapted to be tightened to secure a rod within slot 184.

Clamp ring 141 is sized such that, when relaxed it can slide freely up and down housing 130 of flexible bone anchor 100 and rotate around housing 130. However, when locking set screw 146 is tightened on a rod, clamp ring 141 grips coupling surface 136 of housing 130 and prevents offset connector 140 from moving in any direction. Saddle 143 is pivotably connected to clamp ring 141 by pivot pin 144. Saddle 143 can pivot about pivot pin 144. However, when locking set screw 146 is tightened on a rod, plunger 148 grips clamp ring 141 and prevents further movement of saddle 143. In this way, operation of the single set screw 146 serves to lock the clamp ring 141 to the coupling surface 136 of the flexible bone anchor 100, fix saddle 143 in a fixed position relative to clamp ring 141 and secure a vertical rod within the slot 184 of offset connector 140.

The connector of FIG. 1B is provided by way of example only. It is desirable to have a range of different connectors which are compatible with the anchor system and deflection system. The connectors may have different attributes, including for example, different degrees of freedom, range of motion, and amount of offset, which attributes may be more or less appropriate for a particular relative orientation and position of two bone anchors and/or patient anatomy. It is desirable that each connector be sufficiently versatile to connect a vertical rod to a bone anchor in a range of positions and orientations while being simple for the surgeon to adjust and secure. It is desirable to provide a set of connectors which allows the dynamic stabilization system to be assembled in a manner that adapts a particular dynamic stabilization assembly to the patient anatomy rather than adapting the patient anatomy for implantation of the assembly (for example by removing tissue\bone to accommodate the system). In a preferred embodiment, the set of connectors comprising the connection system have sufficient flexibility to allow the dynamic stabilization system to realize a suitable dynamic stabilization assembly in all situations that will be encountered within the defined patient population. Alternative embodiments of coaxial heads and offset connectors can be found in U.S. patent application Ser. No. 12/566,485, filed Sep. 24, 2009, entitled “Versatile Polyaxial Connector Assembly And Method For Dynamic Stabilization Of The Spine” (Attorney Docket No. SPART-01043US1) which is incorporated herein by reference.

A vertical rod component is adapted to be mounted to mount 114 of flexible post 104. FIG. 1C shows an exploded view of a vertical rod 150. Vertical rod 150 includes an elongated rod 156 which is preferably a 5 mm titanium rod. At one end of rod 156 is a pocket 157. Pocket 157 is shaped to receive a cobalt chrome ball 152. Ball 152 has a central aperture 153 shaped to receive mount 114 of flexible post 104. Aperture 153 passes through the center of ball 152 and is cylindrical or polygonal in section. Ball 152 is received in pocket 157 and then secured in place by race 154. Race 154 and pocket 157 is preferably threaded in order that race 154 is adapted to be secured to rod 156. Race 154 may also be secured to rod 156 by laser welding or other bonding technology. After being secured in pocket 157 by race 154, ball 152 is still free to rotate within pocket 157. A vertical rod having a mobile joint for connecting the vertical rod to a bone anchor is referred to herein as a dynamic vertical rod. Alternative embodiments of dynamic vertical rods can be found in U.S. patent application Ser. No. 12/566,519, filed Sep. 24, 2009, entitled “Dynamic Spinal Rod And Method For Dynamic Stabilization Of The Spine” (Attorney Docket No. SPART-01044USC) which is incorporated herein by reference.

FIG. 1D shows vertical rod 150 mounted to the mount 114 of a flexible post 104 of a flexible bone anchor 100. As shown in FIG. 1D, mount 114 is passed through aperture 153 of ball 152 (not shown). A nut 160 is then secured to mount 114 securing the ball to mount 114. However, vertical rod 150 may still rotate around ball 152 and pivot relative to flexible post 104. Note that a connector 140 such as shown in FIG. 1B may also be mounted to housing 130 to connect flexible bone anchor 100 to a second vertical rod (not shown). Vertical rod 150 is an example of a dynamic vertical rod.

The components of the dynamic stabilization system are adapted to be assembled and implanted in the spine of a patient to provide a multilevel dynamic stabilization assembly which provides dynamic stabilization of the spine and load sharing. FIG. 1E, shows three adjacent vertebrae 191, 192 and 193. As a preliminary step, flexible bone anchors 100a, 100b, 100c, and 100d comprising flexible posts 104a, 104b, 104c and 104d have been implanted in vertebrae 191 and 192 on the left and right sides of the spinous process 194 between the spinous process 194 and the transverse process 195 of each vertebra. In preferred procedures, threaded shaft of bone anchors 120 are directed so that threaded shafts 120 (not shown) are implanted within the pedicles 196 angled towards the vertebral body 197 of each vertebrae. Threaded shaft 120 (not shown) of each flexible bone anchor 100a, 100b, 100c, 100d is fully implanted in the vertebrae 191, 192. In the example shown in FIG. 1E, polyaxial screws 106a, 106b are implanted in the pedicles 196 of vertebra 193. As shown in FIG. 1E, the housings 130a, 130b, 130c, 130d of each flexible bone anchor 100a, 100b, 100c, 100d remain partly or completely exposed above the surface of the vertebrae so a connection system component can be secured to each flexible bone anchor 100a, 100b, 100c and 100d.

After installation of the flexible bone anchors and polyaxial screws, the vertical rod system components and connection system components are adapted to be installed and assembled. FIG. 1E shows, on the right side of the vertebrae, one way to assemble deflection system components and connection system components. Offset heads/connectors are adapted to be externally-mounted to the outside surface of each of housings 130a, 130b, 130c and 130d. An offset connector 140d is shown mounted to housing 130d or flexible bone anchor 100d. A first vertical rod 150c is connected at one end to flexible post 104c by ball-joint 158c. First vertical rod 150c is connected at the other end by offset connector 140d to flexible bone anchor 100d. A second vertical rod 150d is connected at one end to flexible post 104d by ball-joint 158d. Second vertical rod 150d is connected at the other end to polyaxial screw 106b.

The dynamic stabilization assembly 190 of FIG. 1E thus has a vertical rod 150c, 150d stabilizing each spinal level (191-192 and 192-193). Each of the vertical rods 150c, 150d is secured rigidly at one end to a bone anchor (120c, 120d). Each of the vertical rods 150c, 150d is secured at the other end by a ball-joint to a flexible post 104c, 104d thereby allowing for some movement and load sharing by the dynamic stabilization assembly. Offset connector 140d permits assembly of the dynamic stabilization assembly for a wide range of different patient anatomies and/or placements of flexible bone anchors 100a, 100b, 100c and 100d. A similar assembly is preferably implanted on the left side of the spine. FIG. 1F shows a lateral view of the dynamic stabilization assembly 190 of FIG. 1E.

The particular dynamic stabilization assembly shown in FIGS. 1E and 1F is provided by way of example only. An identical or similar dynamic stabilization assembly would preferably be implanted on the left side of the spine. It should be noted that the dynamic stabilization assembly does not require horizontal bars or locking screws thereby reducing the exposure of tissue and/or bone to foreign bodies compared to systems with this additional hardware. The dynamic stabilization assembly thereby, has a small footprint, potentially reducing the amount of displacement of tissue and/or bone, reducing trauma to tissue and/or bone during surgery. Further, the smaller footprint can reduce the amount of tissue that needs to be exposed during implantation. It is an aspect of preferred embodiments of the present invention that the components are adapted to be assembled in different combinations and organizations to create different assemblies suitable for the functional needs and anatomy of different patients. Particular dynamic stabilization assemblies may incorporate various combinations of the bone anchors, vertical rods, flexible bone anchors, offset and coaxial connectors described herein and in the related applications incorporated by reference as well as, in some cases, standard components such as screws, rods and polyaxial screws.

In order to implant the flexible bone anchors 100a, 100b, 100c, 100d, a driver is used to engage the housing 130a, 130b, 130c in order to drive the threaded portion of each bone anchor into the bone. The driver may have a torque-measuring and/or torque limiting function to assist in accurate implantation of the bone screw and avoid excess force being applied to the vertebrae. In alternative embodiments, the flexible bone anchor may incorporate a torque limiting element, for example a secondary head which breaks away when the driver torque exceeds a predetermined torque limit.

### Flexible Bone Anchors

One feature of embodiments of the present invention is the load sharing and range of motion provided by the flexible bone anchors. The flexible bone anchors provide stiffness and support where needed to support the loads exerted on the spine during normal spine motion thereby recovering improved spine function without sacrificing all motion. The flexible bone anchors also isolate the anchor system components from forces exerted by the dynamic stabilization assembly thereby reducing stress on the bone anchors and the bone to which they are attached. Moreover, by selecting the appropriate stiffness of the flexible bone anchor to match the physiology of the patient and the loads that the patient places on the spine, a better outcome is realized for the patient.

As previously described with respect to FIG. 1A, the flexible bone anchor includes a flexible post, and a bone anchor. The flexible post is typically made of biocompatible metal or metals, e.g. titanium and stainless steel. In embodiments of the present invention, the flexible post includes a spring-like flexible section. The spring-like flexible section is more elastic than other regions of the flexible post. The elastic materials of the spring-like flexible section may include biocompatible metals and/or biocompatible polymers. Suitable metals include, for example, titanium, steel and Nitinol. Suitable polymers include, for example, PEEK and Bionate®. The bone anchor secures the flexible bone anchor to the spine. The bone anchor has a threaded shaft connected to a housing which receives the flexible post. The bone anchor is preferably made in one piece from a biocompatible metal, for example, titanium or steel.

The flexible post is configured to connect at one end, to the vertical rod system. The flexible post may deflect relative to the bone anchor by deformation of the flexible post. The deformation of the flexible post imparts force/deflection characteristics to the flexible bone anchor. The movement of the flexible post relative to the bone anchor allows controlled movement of the bone anchor (and vertebra in which it is implanted) relative to the vertical rod system. The flexible bone anchor thus supports the vertebrae to which the bone anchors are attached while allowing movement of the vertebrae thereby providing for dynamic stabilization of the spine.

Flexible bone anchors can be manufactured in a range from highly rigid configurations to very flexible configurations by appropriate selection of the design, materials and dimensions of the flexible post and housing. Flexible bone anchors having a particular stiffness/flexibility can be selected for use in a dynamic stabilization assembly based upon the physiological needs of a particular patient. In a preferred embodiment flexible bone anchor stiffness/flexibility is selected so as to provide load sharing in conjunction with from 50% to 100% of the normal range of motion of a patient and more preferably 70% to 100% of the normal range of motion of a patient.

In some cases, certain of the flexible bone anchors of a dynamic stabilization assembly can have a different stiffness or rigidity or flexibility than other of the flexible bone anchors. Thus, in the same assembly, a first flexible bone anchor can have a first flexibility or stiffness or rigidity, and a second flexible bone anchor can have a second different flexibility or stiffness or rigidity depending on the needs of the patient. Particular embodiments of a dynamic stabilization assembly may utilize flexible bone anchors having different deflection properties for each level and/or side of the dynamic stabilization assembly. In other words, one portion of a dynamic stabilization assembly may offer more resistance to movement than the other portion based on the design and selection of different on the flexible bone anchors having different stiffness characteristics, if that configuration benefits the patient.

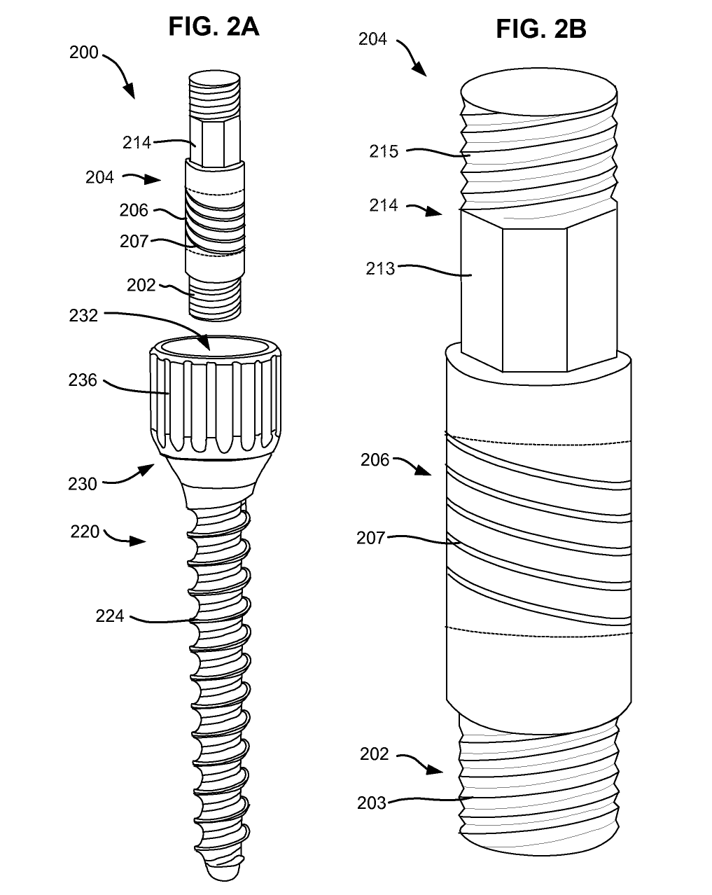

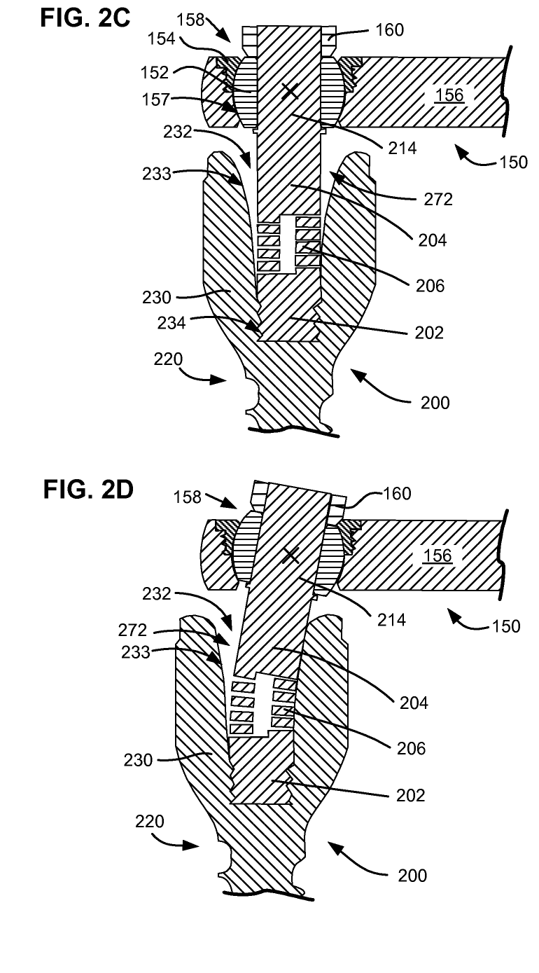

FIGS. 2A through 2D illustrate the design and operation of a first embodiment of a flexible bone anchor 200 including a bone anchor 220 and flexible post 204 according to an embodiment of the present invention. FIG. 2A shows an exploded view of flexible bone anchor 200. Flexible post 204 includes a retainer 202, a flexible section 206 and a mount 214. Mount 214 is designed to connect the proximal end of flexible post 204 to a component of the vertical rod system. In the embodiment shown in FIG. 2A, mount 214 is designed to connect to a dynamic vertical rod (see e.g. dynamic vertical rod 150 of FIG. 1C). Retainer 202 is designed to connect to the distal end of cavity 232.

A flexible section 206 forms part of flexible post 204 between retainer 202 and mount 214. Flexible section 206 is designed to permit movement of mount 214 relative to retainer 202. For example, flexible section 206 may by a portion of flexible post 204 which has enhanced elasticity or flexibility compared to the rest of flexible post 204 by the introduction of a slot or groove 207. Groove 207 has a spiral configuration as shown in the example of FIG. 2B or may have some other configuration adapted to increase the flexibility of flexible post 204. Flexible section 206 is in some embodiments formed in one piece with retainer 202 and mount 214 or may alternatively be formed separately and attached by laser welding, soldering or other bonding technology.

Bone anchor 220 includes a threaded shaft 224 for securing the device to a bone. At the proximal end of the threaded shaft 224 is a housing 230. Housing 230 includes a cavity 232 which is coaxial with the longitudinal axis of the threaded shaft 224. Cavity 232 may, for example, be drilled from one end of flexible post 204. The distal end of the cavity 232 includes a fastener 234 (see FIG. 2B) which engages the retainer 202 of flexible post 204 to secure the flexible post 204 within the cavity 232.

Flexible bone anchor 200 also preferably includes a coupling surface 236 to which other components are adapted to be mounted. As shown in FIG. 2A, coupling surface 236 is the external surface of housing 230. Flexible bone anchor 200 thus provides two mounting positions, one being the mount 214 of flexible post 204 (a coaxial mounting position) and one being the coupling surface 236 (an external or offset mounting position). Thus, a single flexible bone anchor 200 can serve as the mounting point for one, two or more components. For example, a vertical rod may be mounted to mount 214 and a component of the connection system may be mounted to the outer surface 236 of the housing 230 (See, e.g. FIGS. 2C, 2D). As shown in FIG. 2D, mount 214 can deflect relative to bone anchor 220 whereas coupling surface 236 is fixed relative to bone anchor 220. Moreover, housing 230 extends over flexible post 204 to isolate moving parts of flexible bone anchor 200 from the bone. In some embodiments, the flexible bone anchor is adapted to be implanted such that a deflectable portion of flexible post 204 is at or below the surface of the bone.

FIG. 2B shows an enlarged view of flexible post 204. As shown in FIG. 2B, flexible post 204 is generally cylindrical. The proximal end of flexible post 204 includes a mount 214 which includes a polygonal section 213 for receiving a vertical rod and a threaded portion 215 for receiving a nut to secure a vertical rod to the polygonal section 215. The distal end of flexible post 204 includes retainer 202 which has a threaded section 203 for holding the flexible post in a fixed relationship to the bone anchor. Between retainer 202 and mount 214 is flexible section 206 which is generally cylindrical, but includes a groove 207. Groove 207 spirals around flexible section 206 rendering it more flexible than mount 214 and/or retainer 202 despite (in this case) being formed in one-piece and of the same material. In alternative embodiments groove 207 has a different shape/configuration adapted to increase the flexibility of flexible post 204. Groove 207 leaves the material of flexible section 206 in the shape of a coil spring. By changing the dimensions of the flexible section 206 and groove 207, the deflection characteristics of the flexible post 204 can be changed. The stiffness of components of the flexible post can be, for example, increased by increasing the diameter of the post. Additionally, increasing the amount of material removed in groove 207 will decrease the stiffness of the flexible post. Alternatively and/or additionally, changing the materials which comprise the components of the post 204 can also affect the stiffness of the flexible post. For example, making flexible post 206 out of stiffer material reduces deformation of flexible post 204 for the same amount of load—all other factors being equal.

The flexible post 204 may have the same force deflection response in each direction of deflection of the flexible post (isotropic). The flexible post 204 may alternatively have different force/deflection properties in different directions (anisotropic). For example, the flexible post 204 can have different modulus of elasticity in different directions by adjusting, for example, the thickness of the groove 207 in one region compared to another region.

The stiffness of the flexible post may thus be varied or customized according to the needs of a patient. Furthermore, one feature of the present invention is to allow the efficient manufacture of a range of flexible bone anchors having a range of different force-deflection characteristics. This can readily be accomplished by manufacturing a range of flexible posts 204 having different force-deflection characteristics and leaving the remainder of the components unchanged. In this way, the range of flexible bone anchors is adapted to be manufactured with a minimum number of unique parts.

By adjusting the properties of flexible post 204, the deflection characteristics of the flexible bone anchor can be configured to approach the natural dynamic motion of the spine, while giving dynamic support to the spine in that region. It is contemplated, for example, that the flexible bone anchor can replicate a 70% range of motion and flexibility of the natural intact spine, a 50% range of motion and flexibility of the natural intact spine and a 30% range of motion and flexibility of the natural intact spine. In some cases, a kit is provided to a doctor having a set of flexible bone anchors with different force/deflection characteristics from which the doctor may select the flexible bone anchors most suitable for a particular patient. In other cases, the surgeon may select flexible bone anchors prior to the procedure based upon pre-operative assessment.

FIGS. 2C and 2D are section views of flexible bone anchor 200 mounted to a dynamic vertical rod 150. FIGS. 2C and 2D also illustrate deflection of flexible post 204.

Referring now to FIG. 2C, flexible post 204 is positioned within cavity 232 of housing 230. Retainer 202 of flexible post 204 is engaged in a fixed relationship with a retainer 234 at the distal end of cavity 232. Mount 214 extends out of the proximal opening of cavity 232. In an unloaded configuration, flexible post 204 is coaxial with cavity 232 which is coaxial with threaded shaft 224 of bone anchor 220. Towards the proximal end of cavity 232 there is a gap 272 between flexible post 204 and a limit surface 233.

Referring again to FIG. 2C, mount 214 connected to a ball 152 of a dynamic deflection rod 150. Ball 152 is trapped within pocket 157 of vertical rod 150 by race 154 forming a ball-joint 158 which allows vertical rod 156 to rotate 360 degrees around the axis of flexible post 204 and also tilt away from the plane perpendicular to the axis of flexible post 204. Thus, the vertical rod 150 is allowed to rotate and/or have tilting and/or swiveling movements about a center which corresponds with the center of the ball 152 of ball-joint 158.

As shown in FIG. 2D, applying a force/load to through vertical rod 150 to ball-joint 158 causes deflection of flexible post 204 relative to housing 230. Initially, flexible post 204 bends preferentially in flexible section 206. Deflection of flexible post 204 deforms the flexible section 206 such that flexible post 204 moves across gap 272 between the flexible post 204 and limit surface 233 of housing 230. Flexible post 204 exerts a restoring force pushing mount 214 back towards the center position. Thus, flexible post 204 imparts a return force upon mount 214 to counteract the load. The force required to deflect flexible post 204 depends upon the dimensions of flexible post 204, flexible section 206 and housing 230 as well as the attributes of the material of flexible element 206. In particular, the design of flexible element 206 and elements thereof (See FIG. 2B) is adapted to be adjusted to provide the desired force-deflection characteristics.

As shown in FIG. 2D, as successive portions of flexible post 204 come into contact with the limit surface 233 of the housing 230 the stiffness of the flexible post 204 is increased. The effective flexible length of flexible section 206 is reduced making flexible section 206 appear stiffer as flexible post 204 comes into contact with limit surface 233. Additional deflection may cause further elastic deformation of flexible post 204 however, the force required to deflect flexible post 204 increases significantly after contact of flexible post 204 with housing 230. For example, the stiffness may double upon contact of the flexible post 204 with the limit surface 233. In a preferred embodiment, the proximal end of flexible post 204 may deflect from 0.5 mm to 2 mm before making contact with limit surface 233. More preferably, flexible post 204 may deflect approximately 1 mm before making contact with limit surface 233. Accordingly, the shape of the limit surface 233 of the housing 230 provides a deflection guide which cooperates with the flexible post 204 to control and/or limit the amount and location of deflection of the flexible post 204. The flexible post 204 and the limit surface 233 of the housing 230 thereby define the range of motion and the stiffness which are characteristic of the flexible bone anchor 200. By changing the shape of the flexible post 204, including the design and position of flexible element 206, and the shape of limit surface 233 of the housing 230 these characteristics can be changed.

For example, by changing the rate of change of the diameters and/or the diameters of the flexible post 204 and the limit surface 233 of the housing 230 the range of motion and the stiffness which are characteristic of the flexible bone anchor 200 can be changed. The effective stiffness of the flexible bone anchor can be, for example, increased by increasing the diameter of the flexible post and/or by decreasing the diameter of the limit surface 233 of housing 230 as both approach. Additionally, decreasing the diameter of the flexible post will decrease the stiffness of the flexible bone anchor. In addition to changing the dimensions, changing the materials which comprise the components of the flexible post 204 can also affect the stiffness and range of motion of the flexible bone anchor 200.

Thus, the force/deflection response of flexible bone anchor 200 can be customized based on the choice of dimensions and materials. The force deflection characteristics can be configured to approach the natural dynamic motion of the spine, while giving dynamic support to the spine in that region. It is contemplated, for example, that the flexible bone anchor can be made in stiffness that can replicate a 70% range of motion and flexibility of the natural intact spine, a 50% range of motion and flexibility of the natural intact spine and a 30% range of motion and flexibility of the natural intact spine for providing in a kit for a doctor to use.

In a preferred dynamic stabilization assembly incorporating the flexible bone anchor 200, the load sharing and deflection is provided by the flexible bone anchor 200 and to a lesser degree or not in the vertical rod such as the vertical rod 156. It should be noted that ball-joint 158 isolates vertical rod 150 from the torque that would otherwise be placed upon it by the change in angle of mount 214. As load or force is first applied to the vertical rod 150 and the flexible bone anchor 200 by the spine, the deflection of the flexible bone anchor 200 responds about linearly to the increase in the load during the phase when deflection of flexible post 204 causes elastic deformation of flexible element 206. After about 1 mm of deflection, when flexible post 204 contacts limit surface 233 (as shown in FIG. 2D) the flexible bone anchor 200 becomes stiffer. Thereafter, a greater amount of load or force needs to be placed on the flexible bone anchor 200 in order to obtain the same incremental amount of deflection that was realized prior to this point. Accordingly, the flexible bone anchor 200 provides a range of motion where the load supported increases about linearly as the deflection increases and then with increased deflection the load supported increases more rapidly in order to provide stabilization. Put another way, the flexible bone anchor 200 becomes stiffer as the deflection/load increases.

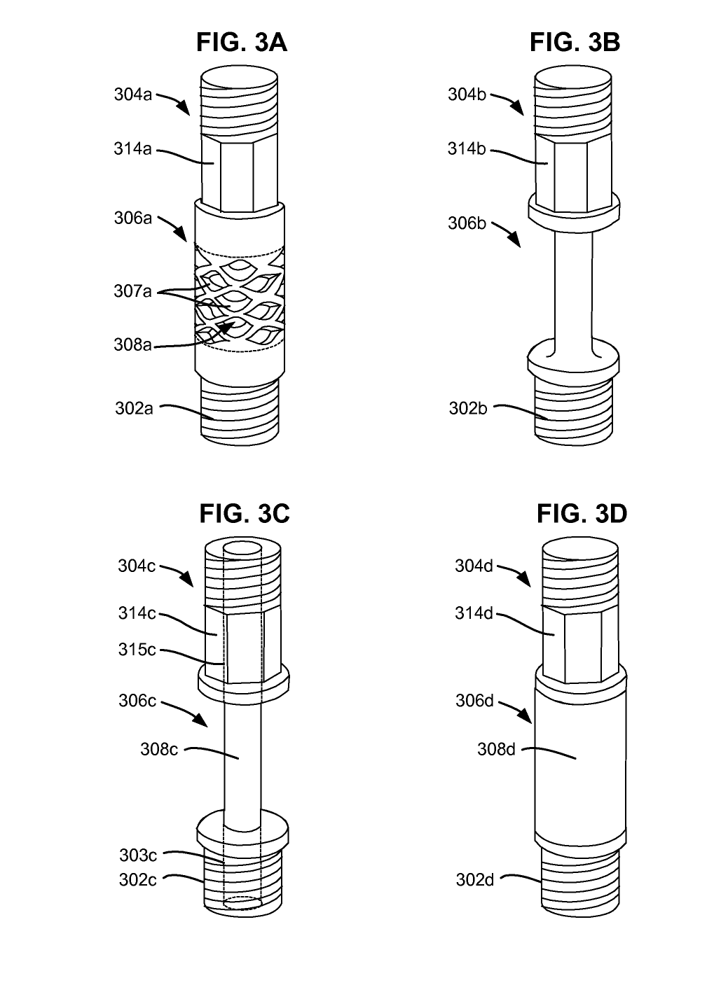

FIGS. 3A-3D show alternative designs for flexible posts which are adapted to be utilized in a flexible bone anchor. FIG. 3A shows a first flexible post 304a. Flexible post 304a includes a mount 314a at the proximal end for connecting to a vertical rod and a retainer 302a at the distal end for connecting in a fixed relationship to a bone anchor. Connected between mount 314a and retainer 302a is a flexible section 306a. Flexible section 306a is cylindrical in shape with an internal cavity 308a. Internal cavity 308a is made, for example, by drilling from one end of flexible post 304a. A plurality of apertures 307a pierces the wall of flexible section 306a into cavity 308a. The apertures 307a are designed to increase the flexibility of flexible section 306a as compared to other regions of flexible post 304a. In the embodiment shown in FIG. 3A, apertures 307a are shaped to leave material of flexible section 306a in the form of a multi-level wave spring. In alternative embodiments, the apertures 307a and cavity 308a are filled with a compliant material. Flexible section 306a is preferably formed in one piece with mount 314a and retainer 302a but may alternatively or may alternatively be formed separately and attached by laser welding, soldering or other bonding technology.

FIG. 3B shows a second flexible post 304b. Flexible post 304b includes a mount 314b at the proximal end for connecting to a vertical rod and a retainer 302b at the distal end for connecting the distal end in fixed relationship to a bone anchor. Connected between mount 314b and retainer 302b is a flexible section 306b. Flexible section 306b is cylindrical in shape but of reduced diameter compared to mount 314b and retainer 302b. The reduction in diameter is designed to increase the flexibility of flexible section 306b as compared to other regions of flexible post 304b. Flexible section 306b is preferably formed in one piece with mount 314b and retainer 302b and of the same material.

FIG. 3C shows a third flexible post 304c. Flexible post 304c includes a mount 314c at the proximal end for connecting to a vertical rod and a retainer 302c at the distal end for connecting the distal end in fixed relationship to a bone anchor. Connected between mount 314c and retainer 302c is a flexible section 306c. Flexible section 306c is cylindrical in shape but of reduced diameter compared to mount 314b and retainer 302b. In the embodiment shown in FIG. 3C, flexible section 306c is a rod 308c of reduced diameter that is formed separately from mount 314c and retainer 302c. Rod 308c are adapted to be received in bores 315c, 303c in mount 314c and retainer 302c in order to connect the parts and attached mechanically, by laser welding, soldering or other bonding technology. Rod 308c is designed to have increased flexibility as compared to other regions of flexible post 304c. Rod 308c is, in some embodiments, formed of the same material as mount 314c and retainer 302c. For example, in one embodiment, rod 308c is formed of titanium/titanium alloy—relying upon reduced diameter for increased flexibility. In another embodiment, rod 308c is formed of a different material than mount 314c and retainer 302c. In another embodiment, rod 308c is formed of a superelastic metal, e.g. nitinol.

FIG. 3D shows a fourth flexible post 304d. Flexible post 304d includes a mount 314d at the proximal end for connecting to a vertical rod and a retainer 302d at the distal end for connecting the distal end in fixed relationship to a bone anchor. Connected between mount 314d and retainer 302d is a flexible section 306d. Flexible section 306d is cylindrical in shape and of substantially the same diameter as mount 314d and retainer 302d. In the embodiment shown in FIG. 3D, flexible section 306d is a rod 308d of substantially the same and formed separately from mount 314d and retainer 302d. Rod 308d is secured to mount 314d and retainer 302d mechanically or by laser welding, soldering or other bonding technology. Rod 308d is designed to have increased flexibility as compared to other regions of flexible post 304d. Rod 308d is in some embodiments formed of a different material than mount 314d and retainer 302d. In some embodiments, rod 308d is formed of a superelastic metal, for example NITINOL.

### Alternative Flexible Bone Anchors

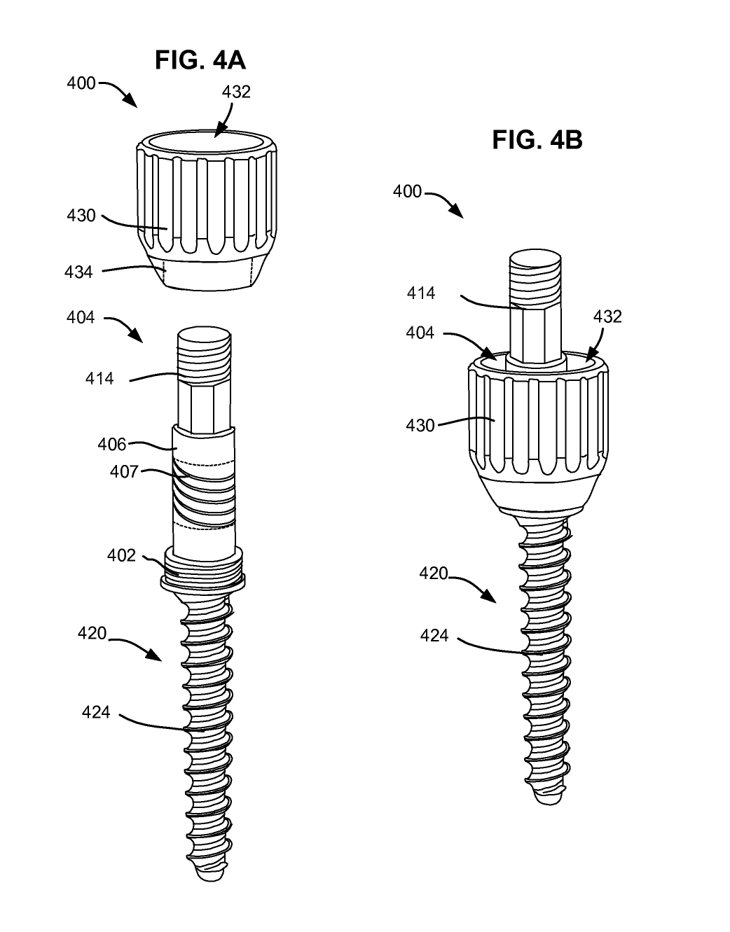

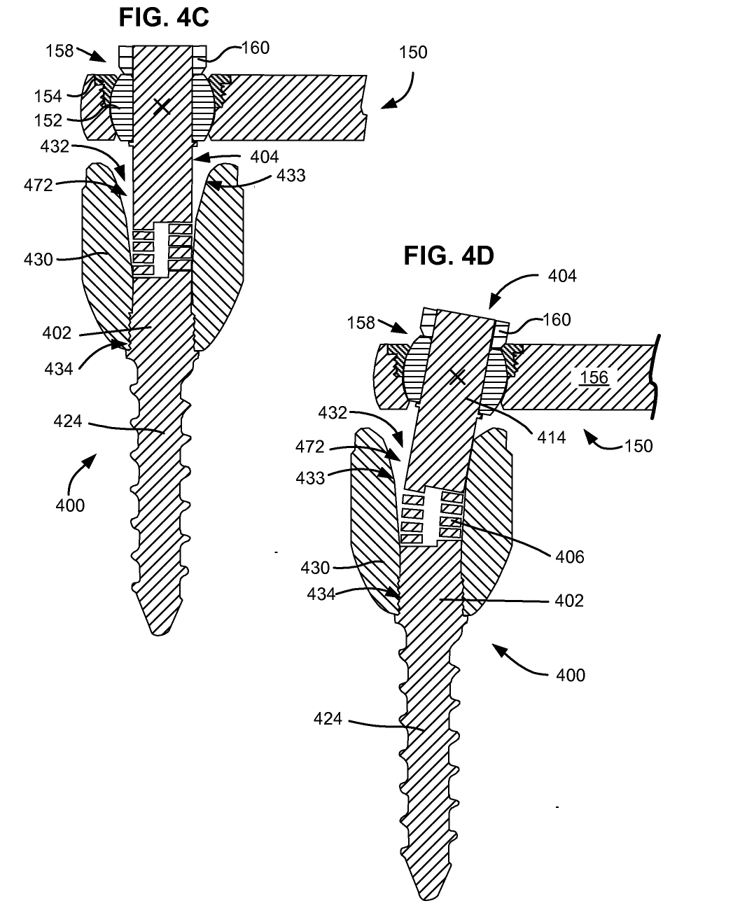

FIGS. 4A through 4C illustrate the design and operation of an alternative embodiment of a flexible bone anchor 400 including a bone anchor 420 and flexible post 404 according to an embodiment of the present invention. FIG. 4A shows an exploded view of flexible bone anchor 400. As shown in FIG. 4A, flexible post 404 includes a retainer 402, a flexible section 406 and a mount 414. Mount 414 is designed to connect the proximal end of flexible post 404 to a component of the vertical rod system. For example, mount 414 is, in some embodiments, adapted to connect to a dynamic vertical rod (see e.g. dynamic vertical rod 150 of FIG. 1C). Retainer 402 is designed to connect the distal end of flexible post 404 in fixed relationship to housing 430. In this embodiment, flexible post 404 is preferably formed in one piece with threaded shaft 424. Threaded shaft 424 is adapted to secure the device to a bone.

A separate housing 430 is provided which can be attached to retainer 402. Housing 430 includes cavity 432 which passes all the way through housing 430 and is aligned with flexible post 404. Flexible post 404 is adapted to be received with cavity 432 of housing 430 and then housing 430 is adapted to be secured in fixed relationship to retainer 402. The distal end of the cavity 432 includes a fastener 434 (see FIG. 4C) which engages the retainer 402 of flexible post 404 to secure the housing 430 to flexible post 404 and threaded shaft 424. Housing 430 may also be attached by laser welding, soldering or other bonding technology.

A flexible section 406 forms part of flexible post 404 between retainer 402 and mount 414. Flexible section 406 is designed to permit movement of mount 414 relative to retainer 402. For example, flexible section 406 may by a portion of flexible post 404 which has enhanced elasticity or flexibility compared to the rest of flexible post 404 by the introduction of a slot or groove 407. Flexible section 406 is preferably formed in one piece with retainer 402, threaded shaft 424 and mount 414 or may alternatively be formed separately and attached by laser welding, soldering or other bonding technology. In some embodiments, flexible section 406 is designed similarly to any one of the flexible sections described herein (See, for example, FIGS. 3A-3D). FIG. 4B shows a perspective view of flexible bone anchor 400, as assembled. Housing 430 has been received over flexible post 404. Retainer 434 has been secured in fixed relationship to retainer 402. Mount 414 extends from the proximal end of cavity 432.

FIGS. 4C and 4D are sectional views of flexible bone anchor 400 mounted to a dynamic vertical rod 150. FIGS. 4C and 4D also illustrate deflection of flexible post 404. Referring now to FIG. 4C, flexible post 404 is positioned within cavity 432 of housing 430. Retainer 402 of flexible post 404 is engaged with fastener 434 at the distal end of cavity 432 of housing 430 to hold the distal end of flexible post 404 in fixed relationship with housing 430. Mount 414 extends out of the proximal opening of cavity 432. In an unloaded configuration, flexible post is coaxial with cavity 432 which is coaxial with threaded shaft 424 of bone anchor 420. Towards the proximal end of cavity 432 there is a gap 472 between flexible post 404 and a contact surface 433.

Referring again to FIG. 4C, mount 414 connected to a ball 152 of a dynamic deflection rod 150. Ball 152 is trapped within a pocket formed by vertical rod 150 and race 154 forming a ball-joint 158 which allows vertical rod 156 to rotate 360 degrees around the axis of flexible post 404 and also tilt away from the plane perpendicular to the axis of flexible post 404. Thus, the vertical rod 150 is allowed to rotate and/or have tilting and/or swiveling movements about a center which corresponds with the center of the ball 152 of ball-joint 158.

As shown in FIG. 4D, applying a force/load to through vertical rod 150 to ball-joint 158 causes deflection of flexible post 404 relative to housing 430. Initially, flexible post 404 bends preferentially in flexible section 406. Deflection of flexible post 404 deforms the flexible section 406 such that flexible post 404 moves across gap 472 between the flexible post 404 and contact surface 433 of housing 430. After further deflection, flexible post 404 comes into contact with limit surface 433 of housing 430. As depicted, the limit surface 433 is configured such that as the flexible post 404 deflects into contact with the limit surface 433, the limit surface 433 is aligned/flat relative to the flexible post 404 in order to present a larger surface to absorb any load an also to reduce stress or damage on the deflectable. Additional deflection may cause further elastic deformation of flexible post 404 however, the force required to deflect flexible post 404 increases significantly after contact of flexible post 404 with housing 430. For example, the stiffness may double upon contact of the flexible post 404 with the limit surface 433. In a preferred embodiment, the proximal end of flexible post 404 may deflect from 0.5 mm to 4 mm before making contact with limit surface 433. More preferably, flexible post 404 may deflect approximately 1 mm before making contact with limit surface 433.

In a dynamic stabilization assembly incorporating the flexible bone anchor 400, the load sharing and deflection is provided by the flexible bone anchor 400 and to a lesser degree or not in the vertical rod such as the vertical rod 150. It should be noted that ball-joint 158 isolates vertical rod 150 from the torque that would other wise be placed upon it by the change in angle of mount 414. As load or force is first applied to the vertical rod 150 and the flexible bone anchor 400 by the spine, the deflection of the flexible bone anchor 400 responds about linearly to the increase in the load during the phase when deflection of flexible post 404 causes elastic deformation of flexible element 406. After about 1 mm of deflection, when flexible post 404 contacts limit surface 433 (as shown in FIG. 4D) the flexible bone anchor 400 becomes stiffer. Put another way, the flexible bone anchor 400 becomes stiffer as the deflection/load increases.

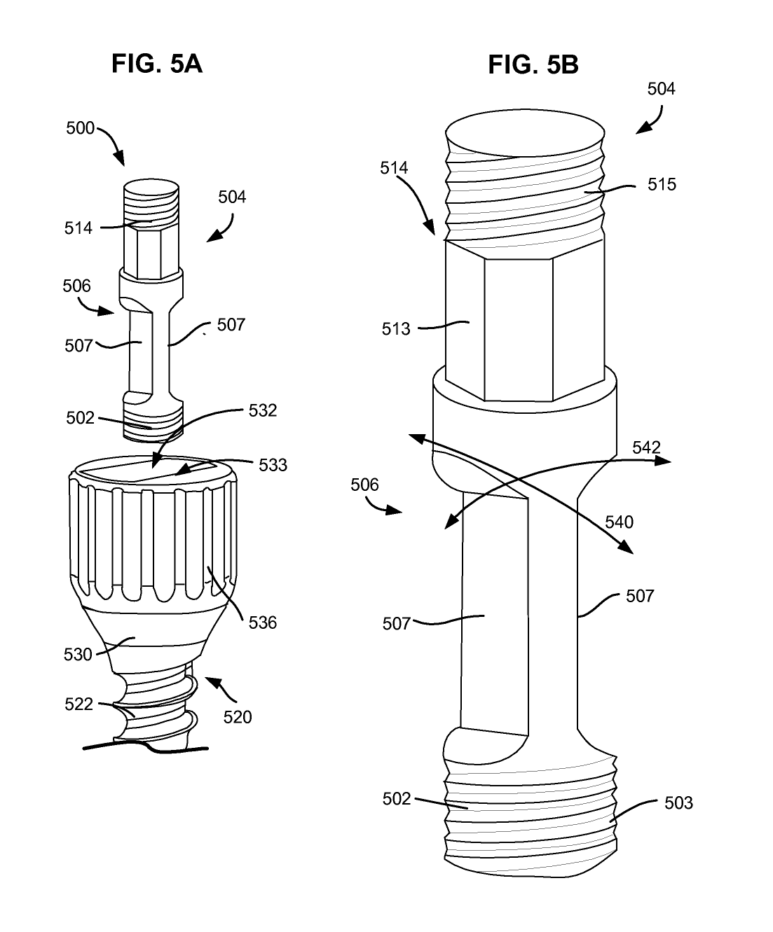

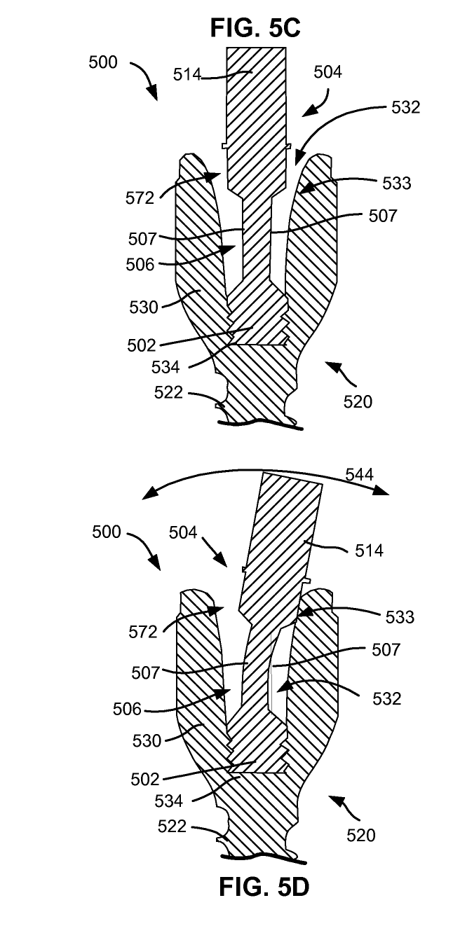

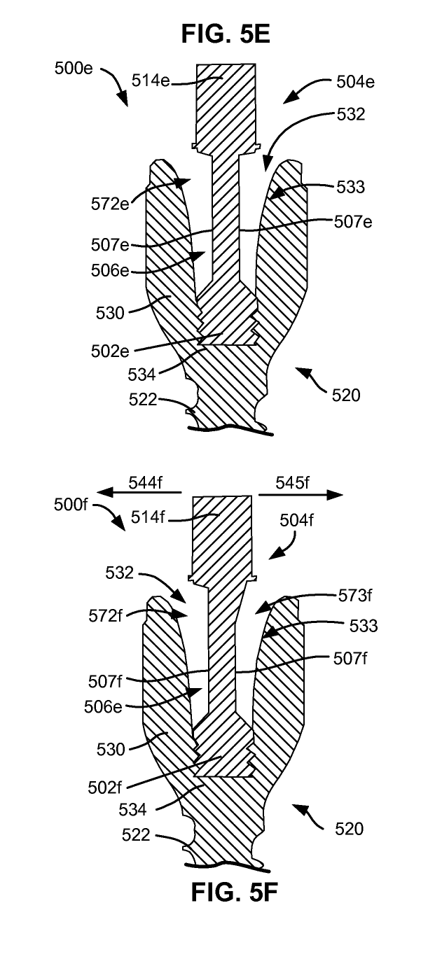

FIGS. 5A-5D show an alternative embodiment of a flexible bone anchor 500. FIG. 5A shows an exploded view of alternative flexible bone anchor 500. Flexible bone anchor 500 includes a flexible post 504 and a bone anchor 520. Flexible shaft 504 includes a proximal mount 514, a distal retainer 502 and a flexible section 506 connecting the proximal mount 514 and distal retainer 502. Bone anchor 520 includes a threaded shaft 522 for engaging a bone and a housing 530 at the proximal end of the threaded shaft 522. The housing 530 has an external coupling surface 536 on which a connector is adapted to be mounted. The housing also has an internal cavity 532 for receiving flexible post 504. Cavity 532 is coaxial with threaded shaft 522. The distal end of the cavity 532 includes a fastener 534 (see FIG. 5C) which engages the retainer 502 of flexible post 504 to secure the distal end of flexible post 504 within the cavity 532 and in fixed relationship thereto.

A flexible section 506 forms part of flexible post 504 between retainer 502 and mount 514. Flexible section 506 is designed to permit movement of mount 514 relative to retainer 502. For example, flexible section 506 may by a portion of flexible post 504 which has enhanced elasticity or flexibility compared to the rest of flexible post 504 by the removal of material from sides 507. Flexible section 506 is preferably formed in one piece with retainer 502 and mount 514 or may alternatively be formed separately and attached by laser welding, soldering or other bonding technology. Flexible section 506 has a rectangular cross-section which is wider in one direction than the other. Flexible section 506 is thus more flexible bending in a direction parallel to the short axis of the rectangular section (see arrow 542) than in a direction parallel to the long axis of the rectangular section (see arrow 540). Thus flexible section has an anisotropic force-deflection profile.

FIG. 5B shows an enlarged view of flexible post 504. The proximal end of flexible post 504 includes a mount 514 which includes a polygonal section 513 for receiving a vertical rod and a threaded portion 515 for receiving a nut to secure a vertical rod to the polygonal section 513. The distal end of flexible post 504 includes retainer 502 which has a threaded section 503 for holding the flexible post in fixed relationship to the bone anchor. Between retainer 502 and mount 514 is flexible section 506 which has a generally rectangular section—material having been removed from sides 507 compared to a cylinder. The flexible post 504 has different force/deflection properties in different directions (anisotropic). The disparity between the thicknesses of the flexible section 506 in one direction compared to another can be used to control the anisotropic force/deflection profile of the post.

By adjusting the properties of flexible post 504, the deflection characteristics of the flexible bone anchor can be configured to approach the natural dynamic motion of the spine, while giving dynamic support to the spine in that region. It is contemplated, for example, that the flexible bone anchor can replicate a 70% range of motion and flexibility of the natural intact spine, a 50% range of motion and flexibility of the natural intact spine and a 30% range of motion and flexibility of the natural intact spine. In some cases, a kit is provided to a doctor having a set of flexible bone anchors with different force/deflection characteristics from which the doctor may select the flexible bone anchors most suitable for a particular patient. In other cases, the surgeon may select flexible bone anchors prior to the procedure based upon pre-operative assessment. The anisotropic force/deflection profile of flexible bone anchor 500 may be useful where it is necessary or desirable to provider greater or lesser load-sharing and/or stabilization on one axis of spinal motion as compared to another.

FIGS. 5C and 5D are sectional views of flexible bone anchor 500. FIGS. 5C and 5D also illustrate deflection of flexible post 504. Referring now to FIG. 5C, flexible post 504 is positioned within cavity 532 of housing 530. Retainer 502 of flexible post 504 is engaged with a retainer 534 at the distal end of cavity 532 in fixed relationship thereto. Mount 514 extends out of the proximal opening of cavity 532. In an unloaded configuration, flexible post 504 is coaxial with cavity 532 which is coaxial with threaded shaft 522 of bone anchor 520. Towards the proximal end of cavity 532 there is a gap 572 between flexible post 504 and a contact surface 533. This gap is, in some embodiments, larger in the preferential bending directions and smaller in the non-preferred bending direction. Thus not only can the flexible post 504 be stiffer in certain directions than other, the range of motion allowed by housing 530 can also be larger in some directions than others.

As shown in FIG. 5D, applying a force/load to mount 514 causes deflection of flexible post 504 relative to housing 530. Initially, flexible post 504 bends preferentially in flexible section 506. Flexible post 504 will also bend preferentially across the short axis of the rectangular section (see arrow 544). Deflection of flexible post 504 deforms the flexible section 506 such that flexible post 504 moves across gap 572 between the flexible post 504 and surface 533 of housing 530. This gap 572 is, in some embodiments, different in different directions. Flexible post 504 exerts a restoring force pushing mount 514 back towards the center position.

As shown in FIG. 5D, after further deflection, flexible post 504 comes into contact with limit surface 533 of housing 530. Limit surface 533 is configured such that as the flexible post 504 deflects into contact with the limit surface 533, the limit surface 533 is aligned/flat relative to the flexible post 504 in order to present a larger surface to absorb any load an also to reduce stress or damage on the deflectable. Additional loading of mount 515 after contact between flexible post 504 and limit surface 533 may cause further elastic deformation of flexible post 504. However, the force required to deflect flexible post 504 increases significantly after flexible post 504 contacts limit surface 533 adjacent the proximal end of housing 530. For example, the stiffness may double upon contact of the flexible post 504 with the limit surface 533. Thus, the force/deflection response and range of motion of flexible bone anchor 500 can be customized based on the choice of dimensions and materials.

For example, FIG. 5E shows a sectional view of an alternative embodiment of a flexible bone anchor 500e which includes the same parts as flexible bone anchor 500 of FIGS. 5A-5D with the exception of flexible post 504e. Referring now to FIG. 5E, flexible post 504e is positioned within cavity 532 of housing 530. Retainer 502e of flexible post 504e is engaged with a retainer 534 at the distal end of cavity 532 in fixed relationship thereto. Mount 514e extends out of the proximal opening of cavity 532. In an unloaded configuration, flexible post 504e is coaxial with cavity 532 which is coaxial with threaded shaft 522 of bone anchor 520. Towards the proximal end of cavity 532 there is a gap 572e between flexible post 504e and a contact surface 533. Note that the gap 572e is larger in this embodiment than the gap 572 of FIG. 5D thus allowing a greater range of motion of deflection before contact between flexible post 504e and contact surface 533 of housing 530. Additional loading may cause further elastic deformation of flexible post 504e, however, the force required to deflect flexible post 504e increases significantly after contact of flexible post 504e with housing 530. For example, the stiffness may double upon contact of the flexible post 504e with the limit surface 533.

The variation in dimensions and materials can also be utilized to generate an anisotropic force/deflection profile and range of motion. For example, FIG. 5F shows a sectional view of an alternative embodiment of a flexible bone anchor 500f which includes the same parts as flexible bone anchor 500 of FIGS. 5A-5D with the exception of flexible post 504f. Referring now to FIG. 5F, flexible post 504f is positioned within cavity 532 of housing 530. Retainer 502f of flexible post 504f is engaged with a retainer 534 at the distal end of cavity 532 in fixed relationship thereto. Mount 514f extends out of the proximal opening of cavity 532. In an unloaded configuration, flexible post 504f is approximately coaxial with cavity 532 which is coaxial with threaded shaft 522 of bone anchor 520. Towards the proximal end of cavity 532 there are gaps 572f, 573f on either side between flexible post 504f and contact surface 533. Note that the gap 572f on one side is larger than the gap 573f because flexible post 504f is asymmetric. Because gap 572f is larger than gap 573f, flexible post 504f can deflect further in direction 544f before contacting contact surface 533 than in direction 545f. Again, the incremental force required to deflect flexible post 504f increases significantly after contact of flexible post 504f with contact surface 533. For example, the stiffness may double upon contact of the flexible post 504f with the limit surface 533. Thus, flexible bone anchor 550f has an anisotropic range of motion/force deflection response. This may be useful, for example, in applications where it is desired to allow more deflection in one direction (e.g. flexion of the spine) than in another direction (e.g. extension of the spine). Where the flexible bone anchor has an anisotropic force/deflection profile and/or range of motion it is useful to provide visible markings associated with the flexible post and/or housing to guide the surgeon as the correct orientation to implant the flexible bone anchor.

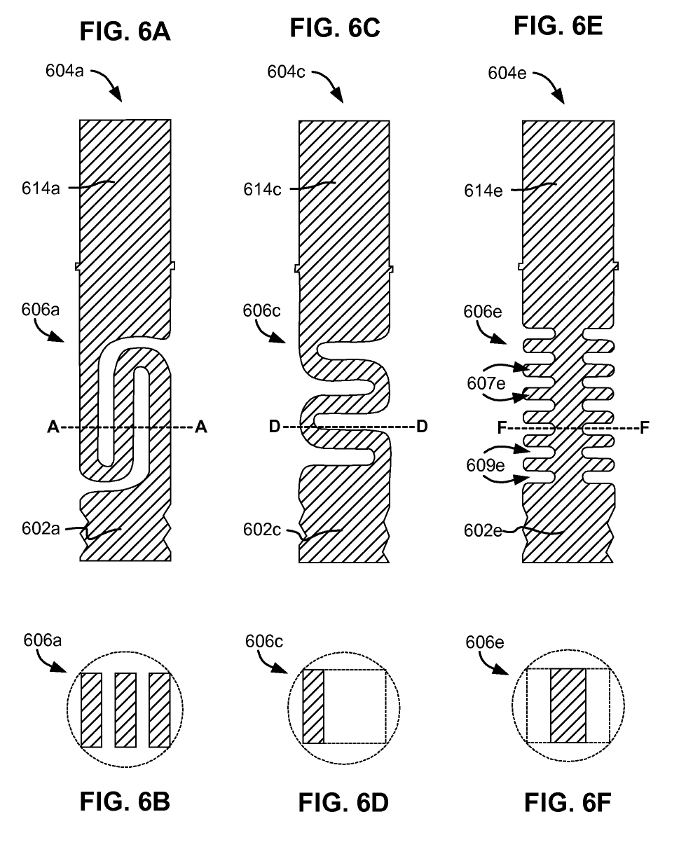

FIGS. 6A-6F show alternative designs for flexible posts having anisotropic force/deflection profiles (i.e. the flexible post is stiffer in some directions than in others). The flexible posts can be adapted for use utilized in the flexible bone anchors previously discussed. FIG. 6A and 6B show sectional views of a first flexible post 604a. FIG. 6A shows a section parallel to the long axis of the flexible post 604a. FIG. 6B shows a section perpendicular to the long axis of the flexible post 604a (see line A-A of FIG. 6A). Flexible post 604a includes a mount 614a at the proximal end for connecting to a vertical rod and a retainer 602a at the distal end for connecting the distal end of flexible post 604a in fixed relationship to a bone anchor. Connected between mount 614a and retainer 602a is a flexible section 606a. Flexible section 606a is rectangular in section and forms a vertical S-shape. The shape allows for a greater length of material within flexible section 606a allowing for enhanced flexibility. As shown in FIG. 6B, the material in flexible section 606a is rectangular in section and thus the flexible post has an anisotropic force/deflection profile. Flexible section 606a is preferably formed in one piece with mount 614a and retainer 602a but may alternatively or may alternatively be formed separately and attached by laser welding, soldering or other bonding technology.

FIGS. 6C and 6D show sectional views of a second flexible post 604c. FIG. 6C shows a section parallel to the long axis of the flexible post 604c. FIG. 6D shows a section perpendicular to the long axis of the flexible post 604c (see line D-D of FIG. 6C). Flexible post 604c includes a mount 614c at the proximal end for connecting to a vertical rod and a retainer 602c at the distal end for connecting the distal end in fixed relationship to a bone anchor. Connected between mount 614c and retainer 602c is a flexible section 606c. Flexible section 606c is rectangular in section and forms a horizontal S-shape. The shape allows for a greater length of material within flexible section 606c allowing for enhanced flexibility. As shown in FIG. 6D, the material in flexible section 606c is rectangular in section and thus the flexible post has an anisotropic force/deflection profile. Flexible section 606c is preferably formed in one piece with mount 614c and retainer 602c but may alternatively or may alternatively be formed separately and attached by laser welding, soldering or other bonding technology.

FIGS. 6E and 6F show sectional views of a third flexible post 604e. FIG. 6E shows a section parallel to the long axis of the flexible post 604e. FIG. 6F shows a section perpendicular to the long axis of the flexible post 604e (see line F-F of FIG. 6E). Flexible post 604e includes a mount 614e at the proximal end for connecting to a vertical rod and a retainer 602e at the distal end for connecting the distal end in fixed relationship to a bone anchor. Connected between mount 614e and retainer 602e is a flexible section 606e. Flexible section 606e is rectangular in section and includes bars 607e extending from the center. The gaps 609e between these bars affect both the force/deflection response and the range of motion. The flexible section 606e becomes stiffer if/when the gaps 609e close during deflection. As shown in FIG. 6F, the principle material in flexible section 606e is rectangular in section and thus the flexible post has an anisotropic force/deflection profile. Flexible section 606e is preferably formed in one piece with mount 614e and retainer 602e but may alternatively or may alternatively be formed separately and attached by laser welding, soldering or other bonding technology.

### Alternative Bone Anchors

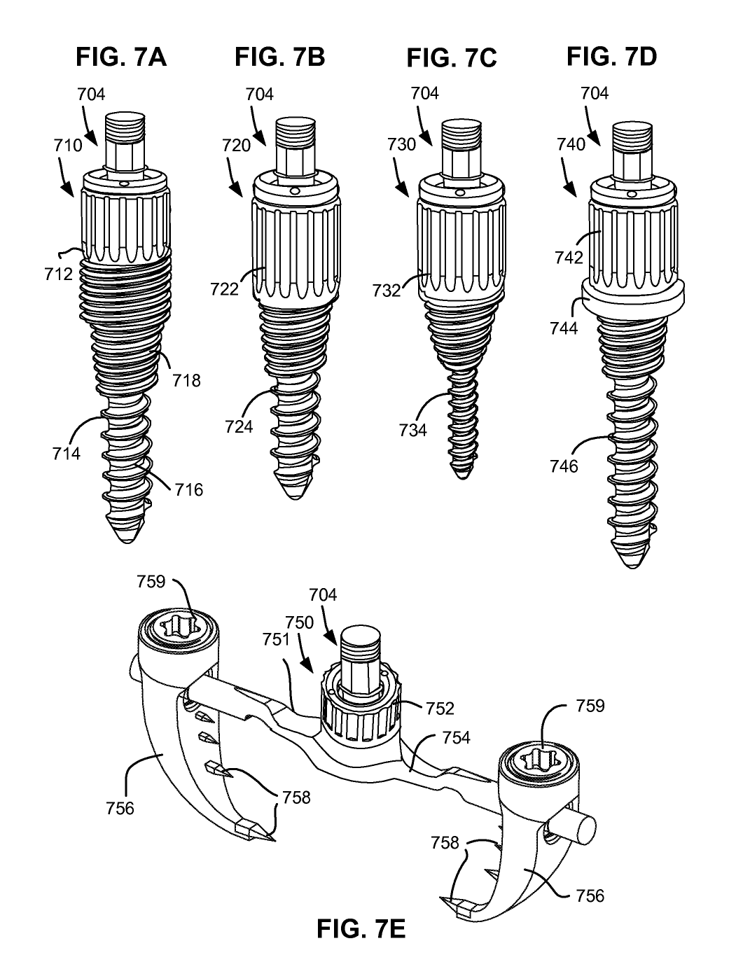

FIGS. 7A through 7E illustrate some possible variations in bone anchors. The bone anchors each have a housing compatible with the flexible posts previously discussed of that can be readily adapted to be compatible. The flexible post is installed/assembled in the bone anchor prior to implantation of the bone anchors in the body. In alternative embodiments, the bone anchors are adapted to be implanted in the body before installation of a flexible post.

Bone anchor 710 of FIG. 7A is a bone screw having a threaded region 714 which extends up over most of a housing 712. A flexible bone anchor 704 is installed in housing 712. The threaded region 714 may extend over a greater or lesser amount of housing 712 depending upon such factors as the length of the bone screw, the type of bone in which the screw is to be implanted and the desired height to which the housing 712 will extend above the bone surface after implantation. Bone anchor 710 advantageously lowers the depth of the pivot point of the flexible bone anchor 704 closer to the natural instantaneous center of rotation of the spine. Note also that the distal thread depth 716 is deeper than the proximal thread depth 718. The distal threads 716 are adapted for engagement of the soft cancellous bone while the proximal threads 718 are adapted for engagement of the harder cortical bone at the surface of the vertebra.

Bone anchor 720 of FIG. 7B is a bone screw in which the screw-only section 724 is shorter in length than in bone anchor 710 of FIG. 7A. A flexible bone anchor 704 is installed in housing 722. Different lengths of screw-only section are useful in different patients or different vertebrae as the size of the bone in which the anchor needs be implanted may vary considerably. For example short bone screws are desirable where the dynamic stabilization system is to be implanted in smaller vertebrae. The physician may determine the length of bone screw appropriate for a particular patient by taking measurements during the procedure by determining measurements from non-invasive scanning, for example, X-ray NMR, and CT scanning Note, however, that housing 722 is preferably the same size and shape as the housings of the other bone anchors to be compatible with the same flexible bone anchors, components and connectors.

Bone anchor 730 of FIG. 7C is a bone screw in which the screw-only section 734 has a smaller diameter and is shorter in length than in bone screw 710 of FIG. 7A. A flexible bone anchor 704 is installed in housing 732. Different diameters of screw-only section are useful in different patients or different vertebrae as the size of the bone in which the anchor needs be implanted may vary considerably. For example, smaller diameter bone screws are desirable where the dynamic stabilization system is to be implanted in smaller vertebrae. The physician may determine the diameter of bone screw appropriate for a particular patient by taking measurements during the procedure by determining measurements from non-invasive scanning, for example, X-ray NMR, and CT scanning Note, however, that housing 732 is preferably the same size and shape as the housings of the other bone anchors so as to be compatible with the same flexible bone anchors, components and connectors.

Bone anchor 740 of FIG. 7D is a bone screw in which the housing 742 has a rim 744 extending away from housing 742 where it transitions to the threaded region 746. A flexible bone anchor 704 is installed in housing 742. Rim 744 may serve to retain an offset head mounted to housing 742 in a way that it can rotate freely around housing 742 during installation. Rim 744 may also serve to widen the contact area between the bone anchor 740 where it meets the bone of the vertebra. This can act as a stop—preventing over-insertion. This can also provide a wide base for stabilizing the housing against lateral motion and torque. Note that housing 742 is preferably the same size and shape as the housings of the other bone anchors to be compatible with the same flexible bone anchors and connectors.

Bone anchor 750 of FIG. 7E illustrates a bone hook device 751 having a housing 752. A flexible bone anchor 704 is installed in housing 752. Bone hook device 751 comprises a bar 754 to which housing 752 is rigidly connected. At either end of bar 754 is a bone hook 756 having a set screw 759 for securing the bone hook 756 to the bar 754. Each bone hook 756 has a plurality of sharp points 758 for engaging and securing the bone hook 756 to a vertebra. During use, the bone hooks 756 are urged towards each other until the sharp points engage and/or penetrate the surface of a bone. Set screws 759 are tightened to secure bone hooks 756 in position relative to bar 754 and thus secure housing 752 relative to the bone. Different arrangements of bone hooks and bars are made suitable for attachment of the housing 752 to different types, sizes, shapes and locations of vertebra. Note that housing 752 is preferably the same size and shape as the housings of the other bone anchors so as to be compatible with the same flexible bone anchors, components and connectors.

### Flexible Bone Anchor/Loading Rod Materials

Movement of the flexible post relative to the bone anchor provides load sharing and dynamic stabilization properties to the dynamic stabilization assembly. As described above, deflection of the flexible post deforms the material of the flexible section. The characteristics of the material of the flexible section in combination with the dimensions of the components of the flexible bone anchor affect the force-deflection curve of the flexible bone anchor. The dimensions and materials are selected to achieve the desired force-deflection characteristics.

By changing the dimensions of the flexible post, flexible section and housing the deflection characteristics of the flexible bone anchor can be changed. The stiffness of components of the flexible bone anchor can be, for example, increased by increasing the diameter of the flexible post. Additionally, decreasing the diameter of the flexible post will decrease the stiffness of the flexible bone anchor. Alternatively and/or additionally changing the materials which comprise the components of the flexible bone anchor can also affect the stiffness and range of motion of the flexible bone anchor. For example, making the flexible section out of stiffer and/or harder material increases the load necessary to cause a given deflection of the flexible bone anchor.

The flexible section can be formed by extrusion, injection, compression molding and/or machining techniques, as would be appreciated by those skilled in the art. In preferred embodiments the flexible section is formed in one piece with the flexible post. However, in some embodiments, the flexible section is formed separately and then fastened or secured to the other components of the flexible post. For example, a fastener or biocompatible adhesive or welding may be used to secure the flexible section to the components of the flexible post.

The flexible post, bone anchor and vertical rods are, in some embodiments, preferably made of biocompatible implantable metals having the desired deformation characteristics—elasticity and modulus. The metal of the flexible post is preferably able to maintain the desired deformation characteristics over the expected lifetime of the component. Thus the metal is preferably durable, resistant to oxidation and dimensionally stable under the conditions found in the human body. In some embodiments the flexible post is made of, titanium, titanium alloy, a shape-memory or super-elastic metal for example Nitinol (NiTi) or stainless steel. In preferred embodiments the flexible post is made of titanium.

The flexible post is in alternative embodiments, preferably made of a biocompatible and implantable polymer having the desired deformation characteristics—elasticity and modulus. The polymer of the flexible post is preferably able to maintain the desired deformation characteristics over the expected lifetime of the component. Thus the polymer is preferably durable, resistant to oxidation and dimensionally stable under the conditions found in the human body. The flexible post and/or flexible section may, for example, be made from a PEEK or a polycarbonate urethane (PCU) such as Bionate®.

In alternative embodiments, other polymers or thermoplastics are used to make the flexible post and/or flexible section including, but not limited to, polyetheretherketone (PEEK), polyphenylsolfone (Rader), or polyetherimide resin (Ultem®), other grades of PEEK, 30% glass-filled or 30% carbon filled, provided such materials are cleared for use in implantable devices by the FDA, or other regulatory body. Glass-filled PEEK is known to be ideal for improved strength, stiffness, or stability while carbon filled PEEK is known to enhance the compressive strength and stiffness of PEEK and lower its expansion rate. Still other suitable biocompatible thermoplastic or thermoplastic polycondensate materials include materials that have good memory, are flexible, and/or deflectable have very low moisture absorption, and good wear and/or abrasion resistance, can be used without departing from the scope of the invention. These include, for example, polyetherketoneketone (PEKK), polyetherketone (PEK), polyetherketoneetherketoneketone (PEKEKK), and polyetheretherketoneketone (PEEKK), and generally a polyaryletheretherketone. Further, other polyketones can be used as well as other thermoplastics.

Still other polymers that can be used in the flexible post and/or flexible section are disclosed in the following documents, all of which are incorporated herein by reference. These documents include: PCT Publication WO 02/02158 A1, dated Jan. 10, 2002 and entitled Bio-Compatible Polymeric Materials; PCT Publication WO 02/00275 A1, dated Jan. 3, 2002 and entitled Bio-Compatible Polymeric Materials; and PCT Publication WO 02/00270 A1, dated Jan. 3, 2002 and entitled Bio-Compatible Polymeric Materials.

The materials of the flexible post and/or flexible section are selected in combination with the design of the flexible bone anchor to create a flexible bone anchor having stiffness/deflection characteristics suitable for the needs of a patient. By selecting appropriate materials and configuration of the flexible post and/or flexible section, the deflection characteristics of the flexible bone anchor can be configured to approach the natural dynamic motion of the spine of a particular patient, while giving dynamic support to the spine in that region. It is contemplated, for example, that the flexible bone anchor can be made in stiffness that can replicate a 70% range of motion and flexibility of the natural intact spine, a 50% range of motion and flexibility of the natural intact spine and a 30% range of motion and flexibility of the natural intact spine. Note also, as described above, in certain embodiments, a limit surface cause the stiffness of the flexible bone anchor to increase after contact between the flexible post and the limit surface.

The foregoing description of preferred embodiments of the present invention has been provided for the purposes of illustration and description. It is not intended to be exhaustive or to limit the invention to the precise forms disclosed. Many embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the claims and their equivalents.

What is claimed is:

1. A spinal implant comprising:
an elongated bone anchor having a threaded shaft;
a housing associated in fixed relationship to one end of the threaded shaft;
the housing having a bore coaxial with the threaded shaft;
the bore having a limit surface;
a flexible post having a distal end connected to a distal end of the bore in fixed relationship to the housing;
the flexible post having a proximal end extending from a proximal end of the bore;
the flexible post being smaller in diameter than at least the bore such that the proximal end of the flexible post is adapted to move relative to the proximal end of the housing in response to a load applied to the proximal end of the flexible post; and
wherein the limit surface of the bore is positioned to contact the flexible post after the flexible post has moved a predefined amount and thereafter reduce the amount of deflection per unit load.

2. The spinal implant of claim 1, wherein the flexible post comprises a flexible section between the distal end and the proximal end wherein the flexible section has enhanced flexibility compared to other portions of the flexible post.

3. The spinal implant of claim 2, wherein the flexible section of the flexible post is positioned within the bore of the housing.

4. The spinal implant of claim 3, wherein the limit surface curves away from the flexible post moving from the distal end of the bore to the proximal end of the bore.

5. The spinal implant of claim 3, wherein the flexible section comprises a spiral groove adapted to enhance flexibility of the flexible section.

6. The spinal implant of claim 3, wherein the flexible section comprises a plurality of apertures adapted to enhance the flexibility of the flexible section.

7. The spinal implant of claim 3, wherein the flexible section comprises a reduced diameter of material compared to other portions of the flexible post adapted to enhance flexibility of the flexible section as compared to other portions of the flexible post.

8. The spinal implant of claim 3, wherein said bone anchor and said housing are made in one piece.

9. The spinal implant of claim 3, wherein said bone anchor and said flexible post are made in one piece.

10. The spine stabilization device of claim 3, wherein said flexible post has an isotropic deflection profile.

11. The spine stabilization device of claim 3, wherein:
the limit surface of the bore is positioned to contact the flexible post after the flexible post has moved a first predefined amount in a first direction; and
the limit surface of the bore is positioned to contact the flexible post after the flexible post has moved a second predefined amount, different than the first predefined amount, in a second direction different than the first direction.

12. A spine stabilization device comprising:
a bone screw having a housing at a proximal end and a distal end adapted to engage a bone;
a bore in said housing coaxial with the bone screw and having an opening at a proximal end of the housing;
a post having a mount at a proximal end, a retainer at a distal end and a flexible section connecting the mount and the retainer;
the retainer being attached to the housing within the bore such that,
the post is coaxial with the bore,
the flexible section of the post is within the bore spaced from the housing, and
the mount extends from the opening of the bore;
whereby application of a transverse load to the mount causes the flexible section of the post to bend allowing the mount to move relative to the housing.

13. The spine stabilization device of claim 12, further comprising a limit surface associated with the housing and positioned to contact the deflectable post after a first amount of bending of the flexible section of the post.

14. The spine stabilization device of claim 13, wherein the post is made in one piece and substantially cylindrical and the flexible section comprises a spiral groove adapted to enhance flexibility of the flexible section.

15. The spine stabilization device of claim 13, wherein the post is made in one piece and substantially cylindrical and the flexible section comprises a plurality of apertures adapted to enhance flexibility of the flexible section.

16. The spine stabilization device of claim 13, wherein the post is made in one piece and substantially cylindrical and the flexible section comprises a reduced diameter of material compared to other portions of the flexible post adapted to enhance flexibility of the flexible section as compared to other portions of the post.

17. The spine stabilization device of claim 13, the flexible section bends a greater amount per unit load prior to contacting the limit surface than subsequent to contacting the limit surface.

18. The spine stabilization device of claim 13, wherein said flexible section has an isotropic deflection profile.

19. The spine stabilization device of claim 13, wherein:
the limit surface of the bore is positioned to contact the flexible post after the flexible post has moved a first predefined amount in a first direction; and
the limit surface of the bore is positioned to contact the flexible post after the flexible post has moved a second predefined amount, different than the first predefined amount, in a second direction different than the first direction.
